(19) United States
(12) Patent Application Publication
(10) Pub. No.: US 2025/0346863 A1
(43) Pub. Date: Nov. 13, 2025

(54) CELL THERAPY COMPOSITIONS AND METHODS OF MANUFACTURE

(71) Applicant: CAPRICOR, INC., San Diego, CA (US)

(72) Inventors: Kristi ELLIOTT, Encinitas, CA (US); Minghao SUN, San Diego, CA (US); Lena TRINH, San Diego, CA (US); Yujia LI, San Diego, CA (US); Firouz MOHSENIAN, San Diego, CA (US); Arjang SALEHI, Los Angeles, CA (US); Victoria KNORZ, San Diego, CA (US); Li-En HSIEH, La Jolla, CA (US)

(21) Appl. No.: 19/191,968

(22) Filed: Apr. 28, 2025

Related U.S. Application Data
(60) Provisional application No. 63/639,674, filed on Apr. 28, 2024.

(51) Int. Cl. C12N 5/077; A61K 35/34; C12N 5/00; C12Q 1/6881; G01N 33/50

(52) U.S. Cl. CPC C12N 5/0657; A61K 35/34; C12N 5/522; C12N 5/562; C12Q 1/6881; G01N 33/5061; C12N 2500/30; C12N 2513/00; C12N 2533/52

(57) ABSTRACT

Disclosed is a method of making and using a therapeutically potent cell for treating degenerative muscle disease. More specifically, disclosed is a method of making and using therapeutic cells, the method including identity and potency release assays for selecting an confirming therapeutic cells useful in ameliorating cardiac muscle and/or skeletal muscle degeneration associated with muscular dystrophy.

### CROSS-REFERENCE TO RELATED APPLICATIONS

This application claims the benefit of priority to U.S. Provisional Application No. 63/639,674 filed on Apr. 28, 2024, the entirety of which is incorporated herein by reference.

### BACKGROUND

Cell and gene therapeutic products are complicated and difficult to manufacture to the exact specifications required for safety and efficacy. Simple and reliable characterization and potency assays that are highly predictive of performance are needed to enable the uniform and standard production of these products. Disclosed are methods of manufacturing cell therapies that include therapeutic cell potency, purity, and identity, which are used in release assays before such cells are used in therapy.

### SUMMARY

In one aspect, a method of manufacturing therapeutic cells is provided. In one embodiment, the method includes growing explants cells from pre-screened heart tissue, producing cardiospheres by culturing the explant cells in media on a low attachment surface, collecting the cardiospheres and then culturing them in media on a high attachment surface to produce cardiosphere-derived cells (CDCs) (a.k.a. CAP-1002 or deramiocel). The CDCs are expanded, collected, and cryopreserved. An aliquot of the cryopreserved CDCs is assessed for identity and therapeutic potency.

In one embodiment, a molecular marker expression assay is used to confirm identity or purity of therapeutic CDCs. The assay includes assessing the expression of IL6, HSPA5, CXCL8, CD105, and CD45.

In another embodiment, an anti-fibrotic collagen expression assay is used to determine or confirm potency of therapeutic CDCs, including assessing the effect of conditioned media from CDCs on the expression of collagen in fibroblasts.

In another embodiment, a beta-catenin production assay is used to determine or confirm potency of therapeutic CDCs.

In another embodiment, an RNA-seq fingerprint assay is used to determine or confirm potency of therapeutic CDCs.

In another embodiment, a relative gene expression profile assay is used to determine or confirm potency of therapeutic CDCs.

In one embodiment, the explant cells are cryopreserved and used as a master cell bank (MCB) for the production of downstream working cell banks or therapeutic cells.

In another aspect, therapeutic cells for use in treating heart or skeletal muscle degeneration are provided. In one embodiment, the therapeutic cells are produced by growing explants cells from pre-screened heart tissue, producing cardiospheres by culturing the explant cells in media on a low attachment surface, collecting the cardiospheres and then culturing them in media on a high attachment surface to produce cardiosphere-derived cells (CDCs). The CDCs are expanded, collected, and cryopreserved. An aliquot of the cryopreserved CDCs is assessed for identity and therapeutic potency.

In one embodiment, a molecular marker expression assay is used to confirm identity or purity of the therapeutic CDCs. The assay includes assessing the expression of IL6, HSPA5, CXCL8, CD105, and CD45.

In another embodiment, an anti-fibrotic collagen expression assay is used to determine or confirm potency of the therapeutic CDCs, including assessing the effect of conditioned media from CDCs on the expression of collagen in fibroblasts.

In another embodiment, a beta-catenin production assay is used to determine or confirm potency of the therapeutic CDCs.

In another embodiment, an RNA-seq fingerprint assay is used to determine or confirm potency of therapeutic CDCs.

In another embodiment, a relative gene expression profile assay is used to determine or confirm potency of the therapeutic CDCs.

In another aspect, a method of treating a degenerative muscle disease in a subject in need is provided. In one embodiment, a dose containing about 150 million therapeutic cells made according to the first aspect are thawed and infused intravenously into the subject once every approximately 3 months.

### DRAWINGS

FIG. 1 is a flow chart depicting certain steps in the production of potent therapeutic cells.

FIG. 2 is a flow chart depicting certain steps in the process of procuring hearts.

FIG. 3 is a flow chart depicting certain steps in the process of creating explants.

FIG. 4 is a flow chart depicting certain steps in the process of outgrowing explant derived cells.

FIG. 5 is a flow chart depicting certain steps in the process of creating a master cell bank from explant derived cells.

FIG. 6 is a flow chart depicting certain steps in the process of forming cardiospheres from explant derived cells or master cell bank cells.

FIG. 7 is a flow chart depicting certain steps in the process of producing and expanding cardiosphere-derived cells from cardiospheres.

FIG. 8 is a flow chart depicting certain steps in the process of harvesting and washing CDCs.

FIG. 9 is a flow chart depicting certain steps in the formulating and packing CDCs.

FIG. 10 is a bar graph depicting percent change in left ventricular ejection fraction (LVEF) as a function treatment of PSBS control, non-potent CDCs, and potent CDCs. CAP-1002 cells (potent CDCs) have a statistically significant, positive ALVEF %, while non-potent cells, including non-potent CAP1002 (CDCs), non-CAP-1002 cells (data not shown), and PBS controls, have a negative ALVEF % as determined by the in vivo mouse MI model (* P<0.01). Only CAP-1002 lots that were classified as potent by this MI mouse model were used clinically.

FIG. 11A is a bar graph depicting percent gene expression of collagen 1A (COL1A; columns 1-4) and collagen 3A (COL3A; columns 5-8 from left to right) as a function of cell line supernatant effect on collagen gene activity in fibroblasts. Columns 1 and 5=non-conditioned media control; columns 2 and 6=conditioned media from CAP-1002 cell-line C; columns 3 and 7=conditioned media from CAP-1002 cell-line B; and columns 4 and 8=conditioned media from CAP-1002 cell-line C. COL1A and COL3A expression analysis was evaluated by qRT-PCR. Assay was performed in triplicate with data shown as average+SD of assay triplicates; all samples had p<0.0001.gene expression analysis was evaluated by qRT-PCR. Assay was performed in triplicate with data shown as average+SD of assay triplicates; all samples had p<0.0001.

FIG. 11B is a bar graph depicting percent gene expression of COL1A as a function of cell line supernatant effect on collagen gene activity in fibroblasts. Column 1=non-conditioned media control; columns 2-10=conditioned media from 9 lots of CDC lines representing six different therapeutically potent CDC lines. Here, therapeutic CDC conditioned media (CM) significantly reduced the type I collagen gene expression (COL1A) in fibroblasts after co-culture. Non-conditioned media (media control) was used as a control. COL1A gene expression analysis was evaluated by qRT-PCR. Assay was performed in triplicate with data shown as average+SD of assay triplicates; all samples had p<0.0001.

FIG. 11C is a bar graph depicting percent gene expression of COL3A as a function of cell line supernatant effect on collagen gene activity in fibroblasts. Column 1=non-conditioned media control; columns 2-10=conditioned media from 9 lots of CDC lines representing six different therapeutically potent CDC lines. Here, therapeutic CDC conditioned media (CM) significantly reduced the type III collagen gene expression (COL3A) in fibroblasts after co-culture. Non-conditioned media (media control) was used as a control. COL3A gene expression analysis was evaluated by qRT-PCR. Assay was performed in triplicate with data shown as average+SD of assay triplicates; all samples had p<0.0001.

FIG. 12A is a bar graph depicting percent inhibition of COL1A gene expression as a function of cell line supernatant effect on collagen gene activity in fibroblasts. Column 1=non-conditioned media control; columns 2-29=conditioned media from 28 lots of CDC lines having proven therapeutic efficacy in human clinical trials. Here, therapeutic CDC conditioned media (CM) significantly reduced the type I collagen gene expression (COL1A) in fibroblasts after co-culture. Non-conditioned media (media control) was used as a control. COL1A gene expression analysis was evaluated by qRT-PCR. The dashed line at 35% inhibition represents the 35% COL1A expression reduction acceptance criteria for therapeutic potency. Assay was performed in triplicate with data shown as average+SD of assay triplicates; all samples had p<0.0001.

FIG. 12B is a bar graph depicting percent inhibition of COL3A gene expression as a function of cell line supernatant effect on collagen gene activity in fibroblasts. Column 1=non-conditioned media control; columns 2-29=conditioned media from 28 lots of CDC lines having proven therapeutic efficacy in human clinical trials. Here, therapeutic CDC conditioned media (CM) significantly reduced the type I collagen gene expression (COL1A) in fibroblasts after co-culture. Non-conditioned media (media control) was used as a control. COL1A gene expression analysis was evaluated by qRT-PCR. The dashed line at 45% inhibition represents the 45% COL3A expression reduction acceptance criteria for therapeutic potency. Assay was performed in triplicate with data shown as average+SD of assay triplicates; all samples had p<0.0001.

FIG. 13 is a bar graph depicting percent beta-catenin protein relative to total protein as a function of cell line. DF=human dermal fibroblasts; A=line A CDCs; B=line B CDCs; C=first lot of line C CDCs; C′=second lot of line C CDCs. Here, CAP-1002 cells had statistically higher levels of β-catenin compared to non-CAP-1002 cells (human fibroblasts). β-catenin was analyzed by ELISA. * p<0.05.

FIGS. 14A-14D are bar graphs depicting relative gene expression as a function of cell type, non-CAP1002 (non-potent CDCs) and CAP1002 (potent CDCs). Here, four genes were identified by RNA sequencing as significantly different between CAP-1002 cells (orange; second bar) and non-CAP-1002 cells (blue; first bar), including IL6, HSPA6, CXCL8, and ATP5MF, and confirmed by quantitative RT-PCR.

FIG. 14A depicts relative expression of IL6.

FIG. 14B depicts relative expression of CXCL8.

FIG. 14C depicts relative expression of HSPA5.

FIG. 14D depicts relative expression of ATP5MF.

FIG. 15 is a bar graph depicting percent correlation by Pearson's Correlation Coefficient of RNAseq fingerprinting of several cells lines to the RNAseq fingerprint of known therapeutically potent CDCs (having demonstrated potency for reducing progression of cardiac and/or muscle degeneration in patients with Duchenne muscular dystrophy). The dashed line represents the acceptance criteria of 91% correlation for CDCs to be considered to be potent. Column 1=HEK293 cells; column 2=mesenchymal stem cells (MSC); and columns 3-30=the same 28 lots of CDC lines having proven therapeutic efficacy in human clinical trials as shown in FIGS. 12A and 12B.

### DETAILED DESCRIPTION

Disclosed is a method for producing therapeutically potent cardiosphere-derived cells (a.k.a. CAP-1002, and deramiocel, which are used interchangeably with CDC and have the same meaning as used herein). Therapeutically potent (as used herein, the term potent is used interchangeably and means the same as therapeutically potent) means being effective at ameliorating or modulating or slowing the degeneration of skeletal and heart muscle function in patients with muscular dystrophy.

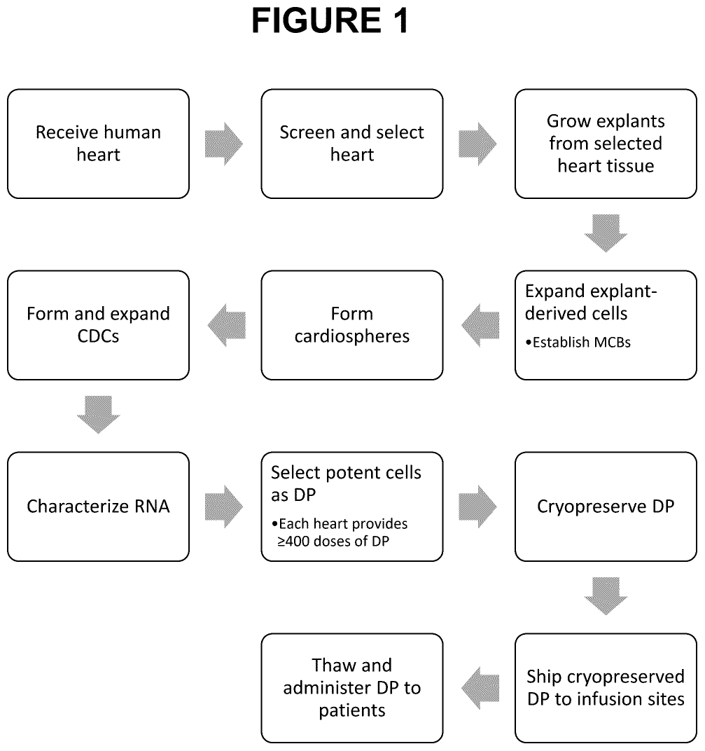

Turning to FIG. 1, in one embodiment, a human heart is offered, screened for appropriate use according to several required attributes, and if the required attributes are present, a heart is selected for further use. The heart tissue is dissected and cultured to allow explant derived cells (EDCs) to grow. The EDCs are expanded in culture, collected, pooled, and then frozen. The frozen EDCs serve as a master cell bank (MCB) for later differentiation and expansion to make the therapeutically potent CDCs. The EDCs (or thawed MCB) are cultured under low attachment conditions to form cardiospheres, which are subsequently collected and transferred to a high attachment tissue culture substrate (plates, flasks, bioreactors, and the like) to promote the formation of cardiosphere-derived cells (CDCs). The CDCs are allowed to form and are expanded by passaging the cells thorough multiple cultures (four to six passages are a preferred embodiment). The expanded CDCs from a given MCB are cryopreserved in both drug product (DP) and quality control (QC) aliquots. A QC aliquot is selected for testing for potency, identity, and purity to permit or not permit the release of the related DP lot. The released DP is then shipped to an infusion site (hospital, clinic, or office where drug is infused into patients), thawed, and administered to patients in need by intravenous infusion. In one embodiment, one dose of drug product includes 150 million therapeutically potent CDCs. In one embodiment, a patient receives one dose every three months to treat skeletal and cardiac muscle degeneration due to muscular dystrophy or other muscle wasting diseases.

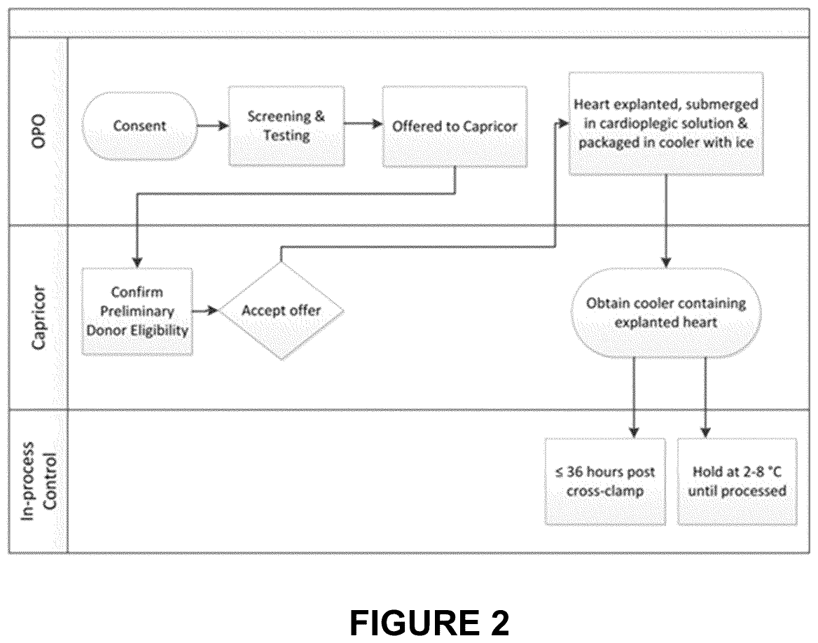

Turning to FIG. 2, in one embodiment, the heart is procured from an organ provider (e.g., organ procurement organization or OPO) who obtains consent from a donor to offer the heart. In order to be eligible for use in the process for making therapeutically potent CDCs, the heart is screened and tested by the OPO according to certain criteria (see Table 1) and additional target screening (see Table 2). Once the heart passes the screening criteria, the heart is offered to the therapeutic cell manufacturer or its agent, who then confirms the donor eligibility prior to accepting the offer of the heart. Upon acceptance of the offer, the OPO explants the heart, packs it in cardioplegic in a cooler with ice or similarly effective coolant. The manufacturer obtains the cooler containing the explanted heart. In-process controls include processing the heart within no more than 36 hours after cross-clamp. The unprocessed heart is held at 2-8° C.

TABLE 1

| Donor | |
|---|---|
| Eligibility | Criteria |
| Inclusion | Inclusion age ≤60 years |
| | Any race |
| | Any gender |
| | Any blood type |
| Exclusion | Drowning submersion time greater than 6 hours |
| | Drowning greater than 60 minutes |
| | Intubation greater than 1 week |
| | Hospitalized greater than 1 month |
| | ICU greater than 2 weeks |
| | IV drug use within 6 months |
| | Positive for any relevant communicable disease agents or |
| | diseases |
| | Sepsis |
| | History of cancer or T. cruzi |

TABLE 2

| Target | Screening test method |
|---|---|
| HIV 1 & 2 | HIV ½ (antibody), HIV 1 (nucleic acid test (NAT)) |
| HBV | HBsAg (surface antigen), HBc IgG (antibody), HBc |
| | IgM (antibody) |
| HCV | HCV (antibody), HCV (NAT) |
| Syphilis | RPR-VDRL (serologic) |
| West Nile Virus | WNV IgG (antibody), WNV IgM (antibody) |
| (WNV)* | |
| TSE, including | Medical history |
| CJD** | |
| Zika Virus (ZKV) | Medical history |
| Vaccinia | Medical history |
| Chagas Disease | Medical history |
| Sepsis | Medical history |
| HLA typing | A, B, BW4, BW6, C, DR, DR51, DR52, DR53, |
| | DQB1, DQA1 (serological) |

*WNV testing is not required for deceased donors (heart donors fall under deceased donor category), however WNV testing may be included in the standard panel of tests performed in accordance with hospital policy (particularly in regions and during seasons where WNV is prevalent) and the testing is done at the discretion of the hospital.
**Transmissible spongiform encephalopathies, including Creutzfeldt-Jakob Disease.

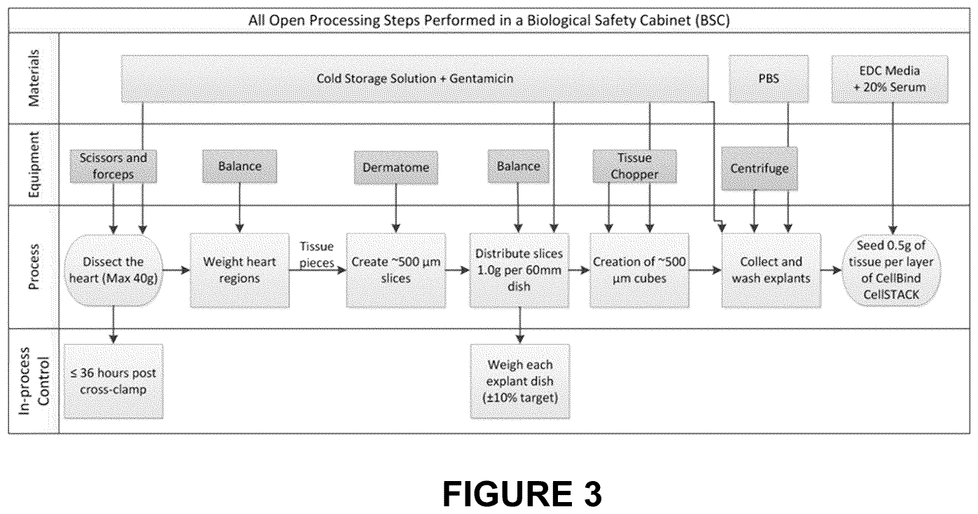

Turning to FIG. 3, explants are made from the accepted heart by processing the heart in cold storage solution plus gentamicin (CSS+G). First, the heart is dissected (within 36 hours post-cross-clamp) and weighed. Tissue pieces are sliced via dermatome into approximate 500-micron slices. The slices are distributed at a rate of about 1 gram per 60 mm dish surface. A tissue chopper is than applied to the slices to make approximate 500-micron cubes. The explant cubes are then collected and washed in phosphate buffered saline (PBS). In one embodiment, The collected and washed explant cubes are seeded at a rate of about 0.5 grams per approximate 500-700 square centimeters of cell culture surface. In one embodiment, collected and washed explant cubes are seeded at a rate of about 0.5 grams per layer of CELLBIND CELLSTACK (Corning, Corning, NY).

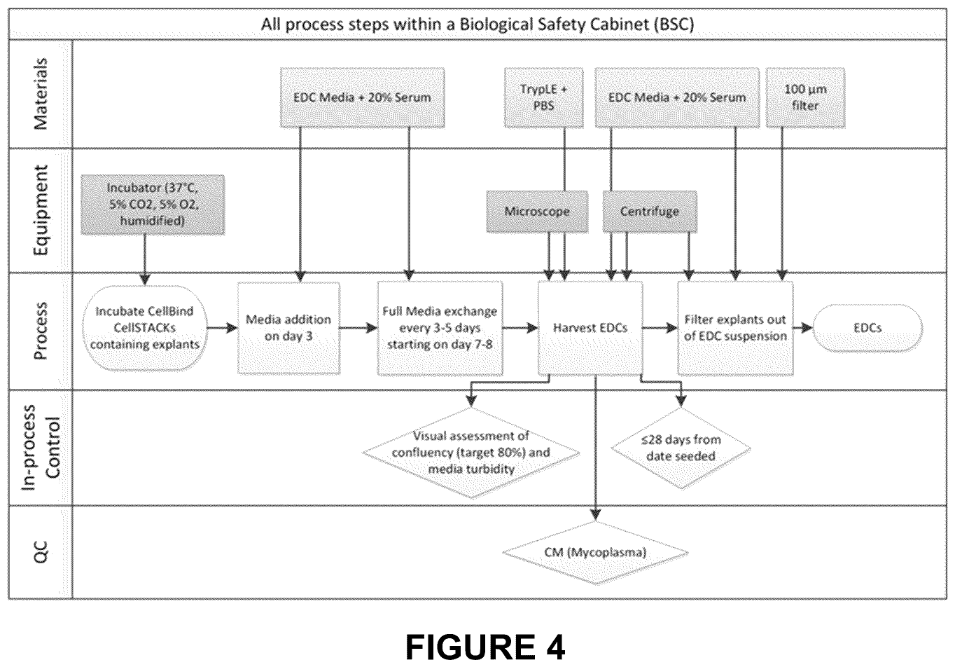

Turning to FIG. 4, the seeded explant cubes are incubated at 37° C. under 5% CO₂ and 5% O₂ in humidified conditions. On about day 3, media containing 20% serum is added to the explant cultures. Starting on or about day 7 or day 8, the media is fully exchanged approximately every 3-5 days or as needed. At about 60%-90% confluency of explant derived cells (EDCs), the EDCs are harvested using a cell dissociation agent, such as trypsin or a serine protease (e.g., TRYPLE, ThermoFisher, Waltham, MA), followed by centrifugation and filtration at 100 microns to remove explants from the EDC suspension.

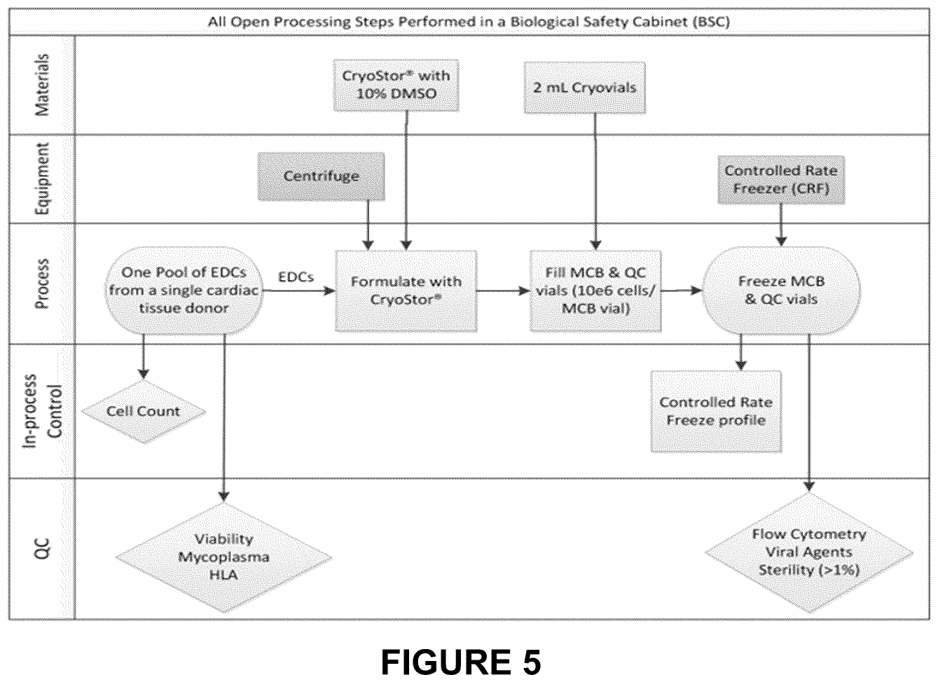

Turning to FIG. 5, a master cell bank (MCB) is made from the EDCs. Here, EDCs from a single heart or single tissue donor are pooled, counted, checked for viability, assessed for HLA type, and checked for Mycoplasma. The pooled EDCs are concentrated (e.g., centrifugation, filtration, or the like) and the formulated with a cryopreservative medium, such as, e.g., 10% DMSO. The formulated pooled EDCs are filled into 2 mL cryovials at about 1,000,000 cells per vial. Some vials (aliquots) are reserved for quality control (QC) and the remainder as mater cell bank (MCB). The QC and MCB vails are frozen by controlled rate. A QC aliquot is checked for identity by flow cytometry, viral agents, and sterility.

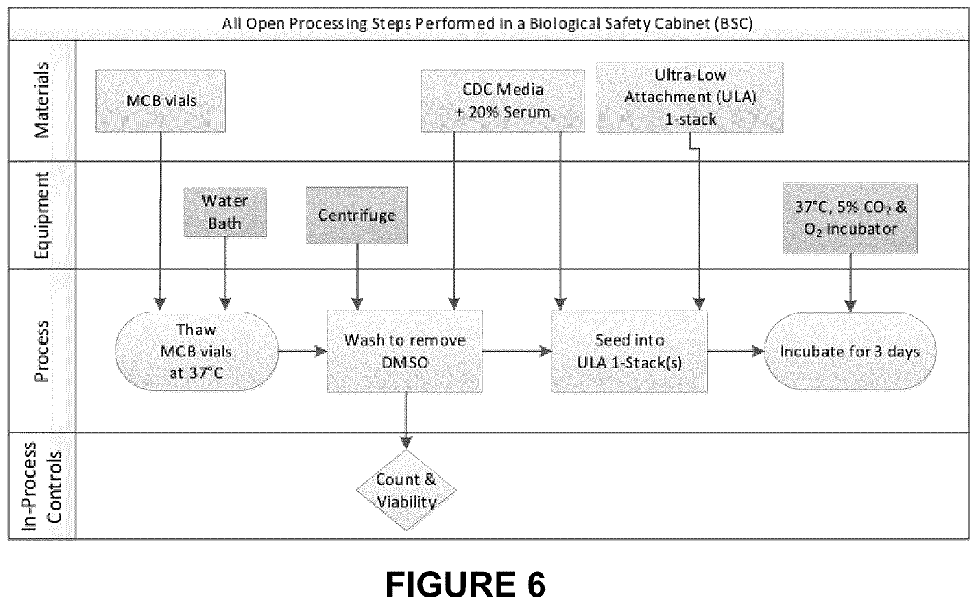

The MCBs are used to produce therapeutically potent CDCs. Turning to FIG. 6, cardiospheres are made from EDCs. Here, MCB vials are thawed, preferably at 37° C. and then washed in media with 20% serum to remove the cryopreservative. In some embodiments, the cells are counted and assessed for viability. The thawed EDCs are seeded onto a ultra-low attachment (ULA) surface (e.g., CORNING ULTRA-LOW ATTACHMENT SURFACE, Corning, Corning, NY) at 37° C., 5% CO₂, and 5% O₂ and incubated for about three days. Cardiospheres develop in the low attachment conditions and are recovered from the cell media supernatant.

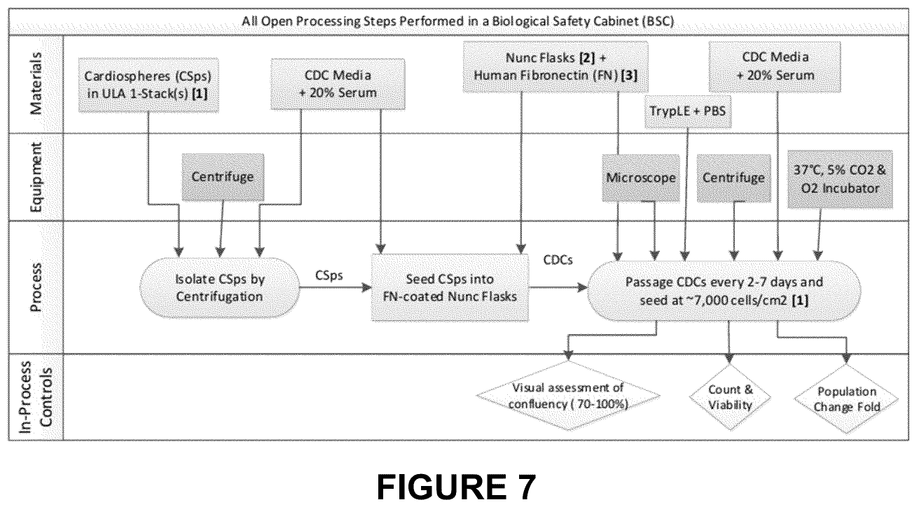

Cardiospheres give rise to cardiosphere-derived cells (CDCs) when plated onto a surface that permits cell attachment. Turning to FIG. 7, cardiospheres are isolated from the ULA cell culture by centrifugation (or filtration or the like) of the decanted media supernatant. The recovered cardiospheres are seeded onto a high attachment surface, such as, e.g., fibronectin-coated flasks (e.g., Nunc Triple Flasks, ThermoFisher, Waltham, MA). CDCs begin to grow out. The cultures are visually assessed for confluency, and at about 70%-100% confluency, the CDCs are passaged. Passaging is done about every 2-7 days and the CDCs are seeded at about 7,000 cells per square centimeter at 37° C. under 5% CO₂ and 5% O₂. Cell viability and counts are monitored. Here, the initial passage (i.e., passage 0 cultures-passage 1 harvest), in which the CDCs are first growing out of the harvested cardiospheres, may require a longer duration (e.g., about 7 days) compared to subsequent passages. In one embodiment, subsequent passages (i.e., passage 2 through passage 5) are limited to a maximum of 5 days of incubation.

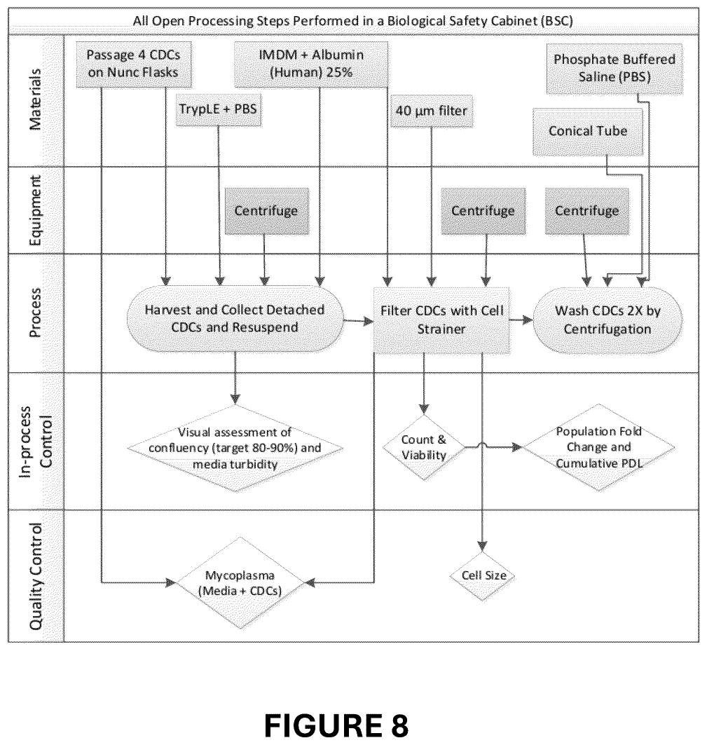

At about 5, the CDCs are harvested and formulated. Turning to FIG. 8, at passage 4 and at about 80%-90% confluency, CDCs are harvested using detachment agent (e.g., trypsin, serine protease, or the like) followed by collection and resuspension in media with 25% albumin. The CDCs are then filtered over a 40 micron filter, counted, and assessed for size and viability, and concentrated. The CDCs are then washed at least twice with PBS by centrifugation (or filtration, or other equivalent means).

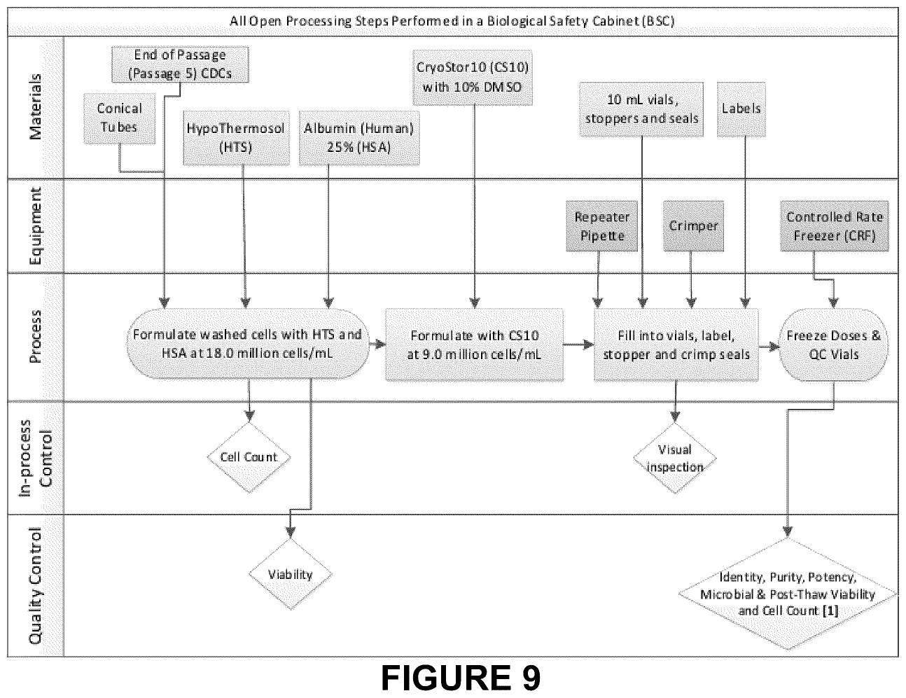

Turning to FIG. 9, the washed CDCs (end of passage or passage 5) are resuspended in hypothermic solution comprising one or more of a buffer, a sugar, a sugar alcohol, glutothione, one or more free-radical scavengers (HTS) (e.g., HYPOTHERMOSOL, BioLife Solutions, Inc., Bothell, WA) and about 25% albumin at a cell concentration of about 18 million cells per milliliter. Cryopreservative containing about 10% DMSO (e.g., CRYOSTOR 10, BioLife Solutions, Inc., Bothell, WA) is added to the cell suspension to a final cell concentration of about 9 million cells per milliliter. The formulated CDCs are loaded into 10 ml vials, stoppered and crimp sealed, and visually inspected. Some aliquots are QC aliquots, and the remainder are drug product doses. The QC aliquots are tested for identity and purity, potency, cell count, post-thaw viability, and contamination.

Table 3 summarizes the release criteria performed on the QC aliquots representing the cryopreserved drug product doses.

TABLE 3

| Parameter | Test Method | Test | Acceptance Criteria |
|---|---|---|---|
| Identity | qRT-PCR | IL6, HSPA5, CXCL8 | IL6: ≥140 fold increase compared to |
| | | | reference human dermal fibroblasts |
| | | | (HDF) mRNA |
| | | | HSPA5: ≥5 fold increase compared to |
| | | | reference HDF mRNA |
| | | | CXCL8: ≥20 fold increase compared |
| | | | to reference HDF mRNA |
| | Flow cytometry | CD105 antigen | >90% CD105 expression cells |
| Viability | Hemocytometer and trypan | Cell viability | ≥70% |
| | blue exclusion | Viable cell number | Report value |
| Potency | mRNA sequencing and | Clinical correlation by | Pearson's Correlation Coefficient: |
| | bioinformatics | RNAseq and bioinformatics | >91% |
| | qRT-PCR | Anti-fibrosis assay | COL1A inhibition: ≥35% compared |
| | | | to non-conditioned media |
| | | | COL3A inhibition: ≥45% compared |
| | | | to non-conditioned media |
| Purity | Flow cytometry | CD45 antigen | <10% CD45 expression cells |
| | Limulus amebocyte lysate | USP <85> endotoxin | <5 EU/kg/hr |
| | (LAL) kinetic chromogenic | | |
| | assay | | |
| | Endosafe endotoxin testing | USP <85> endotoxin | <5 EU/kg/hr |
| | using Nexgen PTS- | | |
| | cartridge technology | | |
| Microbial | Direct inoculation | USP <71> sterility, 14 day | No growth |
| | BACT/ALERT | Sterility | No growth |
| | PCR | Mycoplasma | Negative |

Potency testing had historically been performed on clinical lots of CAP-1002 (a.k.a. CDCs or deramiocel), using an in vivo mouse model of myocardial infarction (MI). Data from this model had been used to select CAP-1002 lots and CAP-1002 master cell banks (MCBs) used in derivation of those lots to support clinical programs, such as HOPE-2 (see McDonald et al., The Lancet, Volume 399, Issue 10329, p 1049-1058 Mar. 12, 2022) and HOPE-2 open label extension (OLE).

Briefly, the in vivo MI mouse model utilizes a minimum of 14 SCID beige mice/group (8-12 weeks, 25-30 g) which each receive a lateral thoracotomy where the left anterior descending coronary artery is permanently ligated to create a MI. CAP-1002 cells or a PBS control are then injected into the border zone of the infarct and mice are sutured and allowed to recover. Echocardiography was performed the day immediately following surgery for a baseline ejection fraction (left ventricle ejection fraction, LVEF) measurement and three weeks post-surgery for a final LVEF measurement.

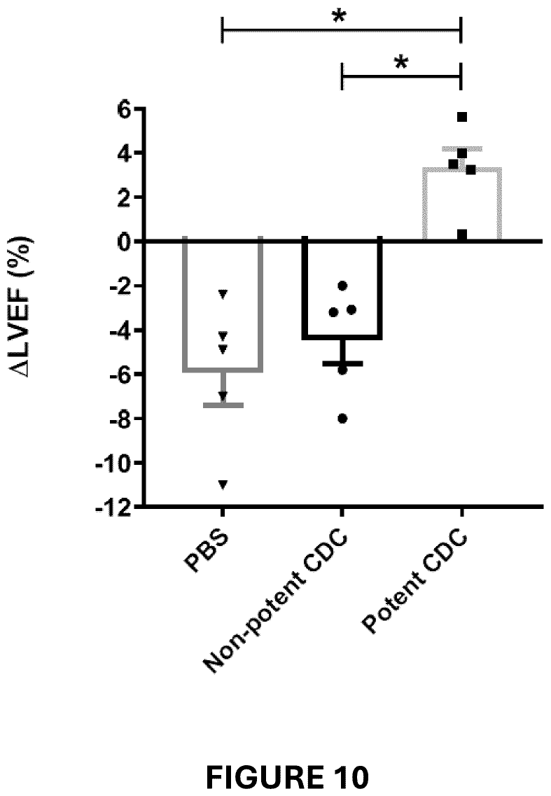

Echocardiographic data was analyzed by two independent reviewers and the change in ejection fraction (ALVEF %) is calculated by subtracting the LVEF % at three weeks from the LVEF % at baseline for each animal. The average change in ejection fraction (ALVEF %) is calculated for control (PBS) and CAP-1002 treated groups. Statistical significance between control and PBS is calculated and if statistical difference is found (p<0.1), a positive ALVEF % is indicative of CAP-1002 potency (FIG. 1). If no statistical difference is found or a negative ALVEF % is recorded, this is indicative of a non-potent CAP-1002 lot (FIG. 10). Only CAP-1002 lots that were classified as potent by this MI mouse model were used clinically. See Smith et al., “Regenerative Potential of Cardiosphere-Derived Cells Expanded From Percutaneous Endomyocardial Biopsy Specimens,” Circulation 115, no. 7 (2007): 896-908, which is herein incorporated by reference for its description of the MI mouse model.

While the MI mouse model has been used as a potency assay to support CAP-1002 clinical programs, including as a model for CAP-1002 as a cell therapy for Duchenne muscular dystrophy (DMD), it is a laborious model requiring months to prepare, execute, analyze, and determine potency for each CAP-1002 lot and thus is not practical for routine QC testing and lot release. Highly skilled surgeons, animal technicians, and scientists are required to consistently execute the assay and analyze the data, which can be subjective. Due to the aforementioned challenges, it is practical for only one lot of CAP-1002 cells from each master cell bank (MCB) to be evaluated by the in vivo MI mouse model for potency with additional lots made from the same MCB assumed as potent or non-potent based on the results from a single lot.

Here, the MI-tested potent CAP-1002 lots, and those CAP-1002 lots derived from the same MCB lots from which the MI-potent CAP-1002 lots were derived, and which were demonstrated to be clinically potent in the HOPE-2 trial and subsequent open label extension, served as benchmarks or positive controls in the disclosed in vitro potency assay.

As described above, an anti-fibrosis assay was used as a potency confirming release assay. Fibrosis is a clear pathological feature observed in muscle from patients with DMD. Fibrosis is defined as tissue hardening with scar formation that results from increased deposition of extracellular matrix proteins, such as collagen. Thus, CAP-1002 may play a role in ameliorating fibrosis. To evaluate anti-fibrotic activity, an in vitro assay was developed using a co-culture system of fibroblasts with conditioned media (CM) collected from CAP-1002.

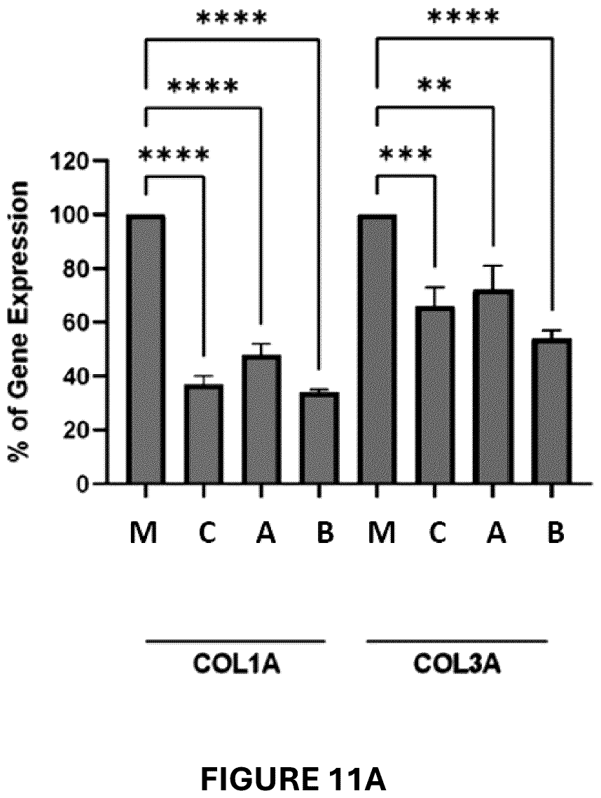

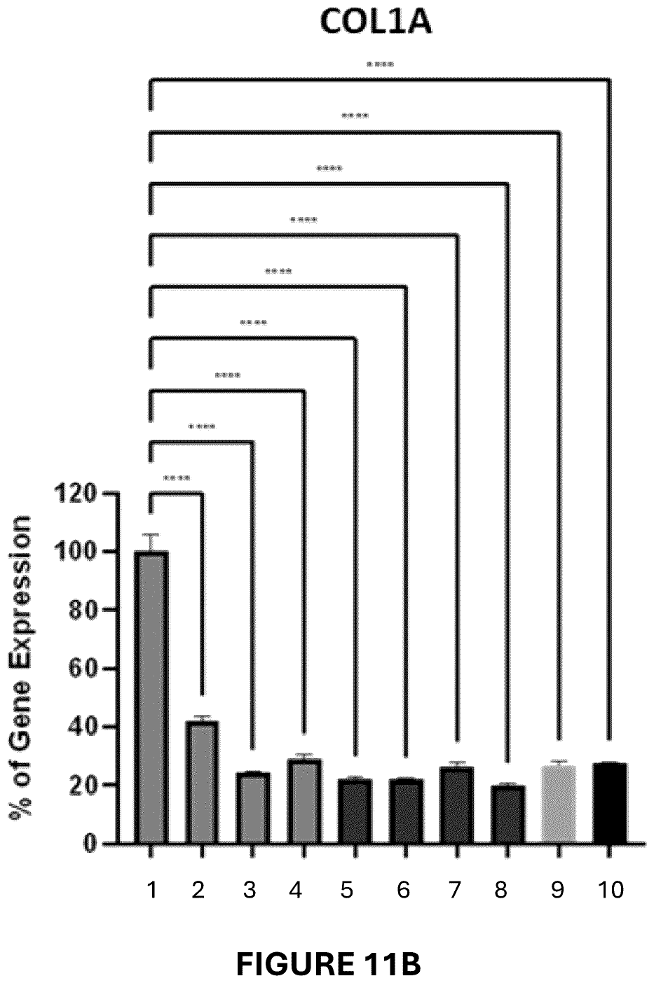

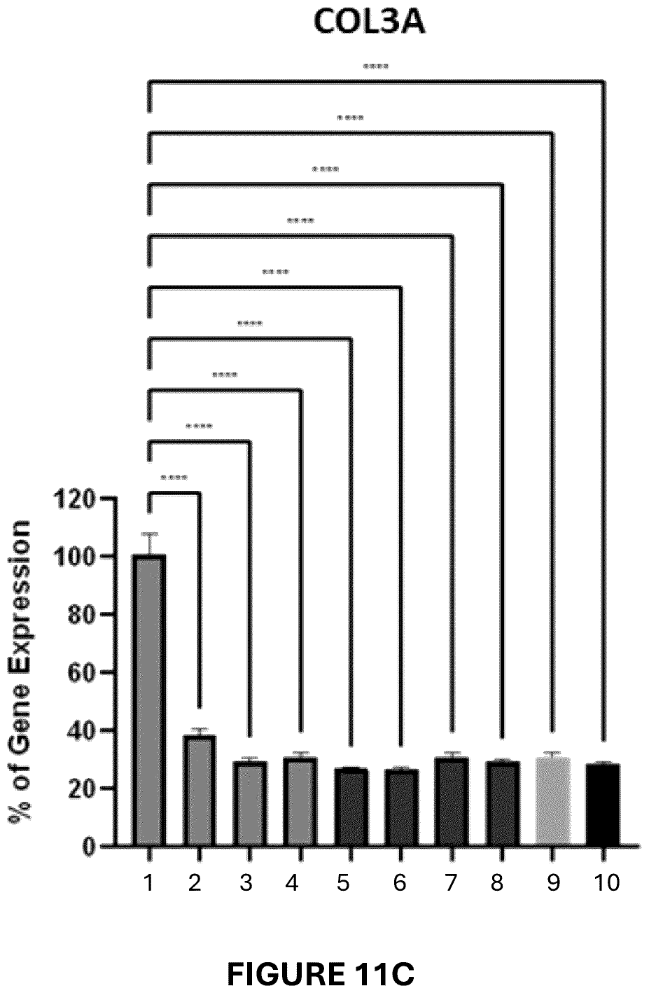

Briefly, human fibroblasts were cultured for 72 hours with CM collected from three different CAP-1002 lots or with non-conditioned media as a control. CAP-1002 lots used in initial development of the assay included line A, line B, and line C. Collagen 1A (COL1A) and collagen 3A (COL3A) expression following fibroblast co-culture with CM was evaluated by qRT-PCR. As shown in FIG. 11A, CM collected from all three lines of CAP-1002 induced a statistically significant reduction in both COL1A and COL3A expression when compared to the non-conditioned media control. Importantly, lines B and C, which utilized in HOPE-2 and HOPE-2 OLE, were classified as potent in the mouse MI model and line A was shown to be effective clinically. Thus, the anti-fibrotic activity as demonstrated by the reduction in collagen expression induced by these two CAP-1002 lines is consistent with potency classified by the in vivo MI mouse model and with clinical potency.

FIGS. 11B and 11C demonstrate significant (p<0.001) reduction in fibroblast COL1A and COL3A expression from conditioned media obtained from 9 lots representing 5 CDC MCB lines relative to media control.

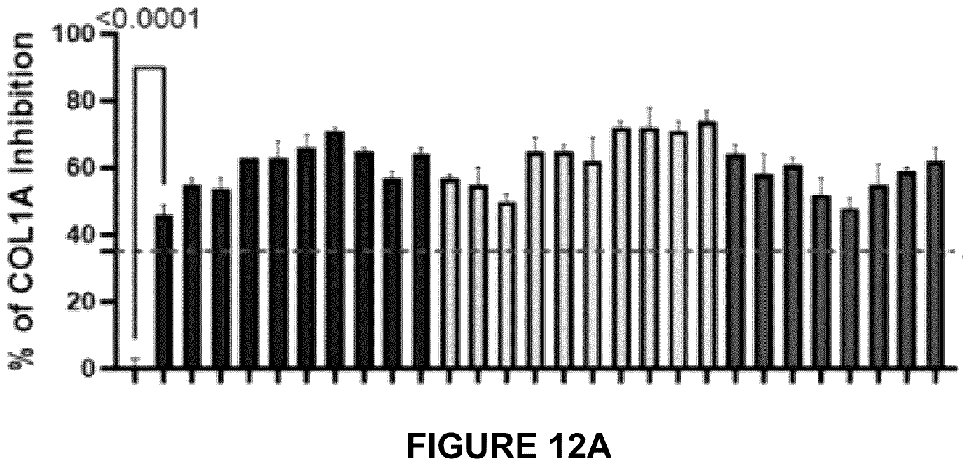

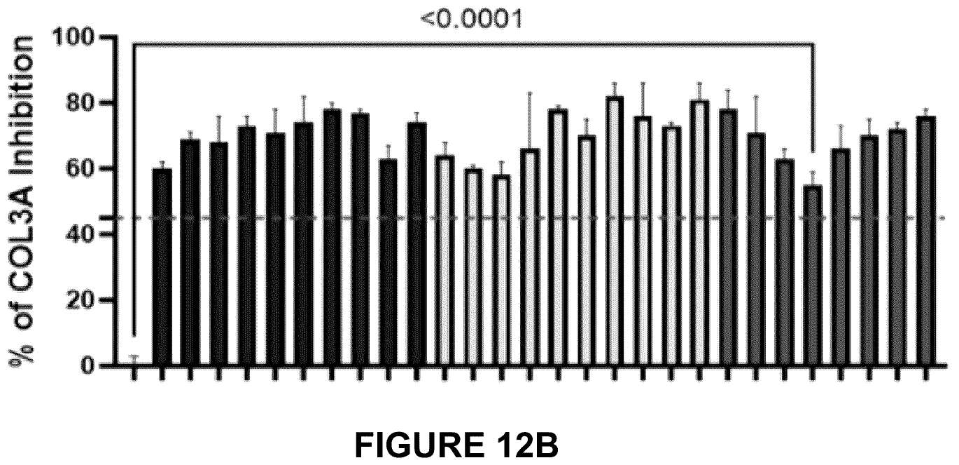

FIGS. 12A and 12B demonstrate that conditioned media from 28 lots representing 5 CDC MCB lines reduced fibroblast COL1A expression by at least 35% relative to non-conditioned media control (the threshold acceptance criterion) and fibroblast COL3A expression by at least 45% relative to non-conditioned media control (the threshold acceptance criterion), respectively.

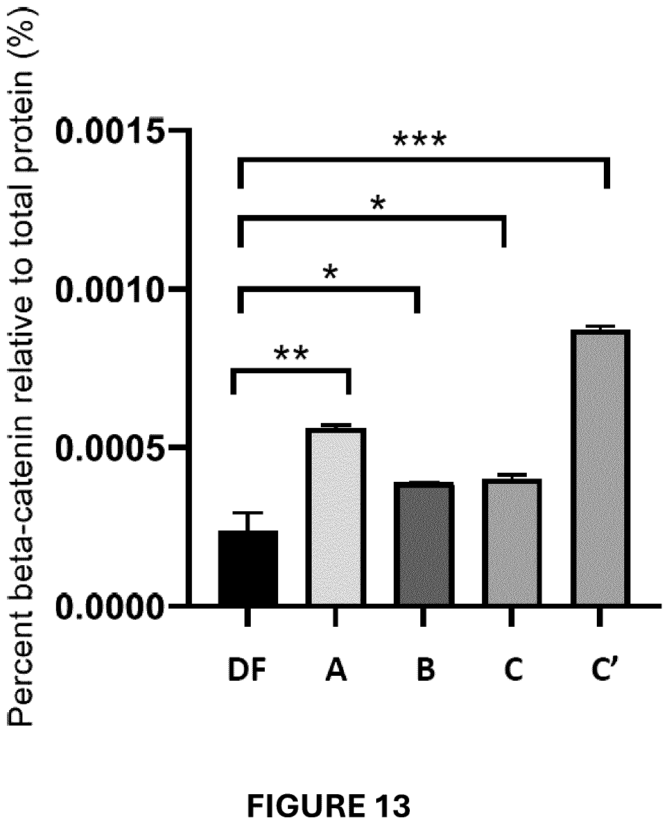

In some embodiments, relative expression of β-catenin may be used to confirm the therapeutic potency of CDCs. β-catenin may play a role as a regulator of several potential mechanisms of action for CAP-1002. β-catenin-dependent Wnt signaling has been implicated in numerous cellular processes, including cell regeneration, cell polarity, and immunomodulation, known to be impacted by CAP-1002. The β-catenin levels of CAP-1002 cells and non-CAP-1002 cells were evaluated by ELISA. Briefly, protein was isolated from three different CAP-1002 lines (lines A-C) and one non-CAP-1002 cell type (human fibroblasts) and analyzed by BCA assay for total protein concentration and by ELISA for β-catenin levels. β-catenin concentration was normalized to the total protein concentration of each lot/type of cell evaluated. CAP-1002 lots used in development of the assay included lines A, B, and C as described above. Line C included two lots, C and C′. As shown in FIG. 13, CAP-1002 cells had statistically higher levels of β-catenin compared to non-CAP-1002 cells (human fibroblasts). When compared to fibroblasts in the assay, CAP-1002 cells had 1.7-3.7× higher β-catenin levels (fold change data not shown). Importantly, the two CAP-1002 lots utilized in HOPE-2 and HOPE-2 OLE (lines B and C/C′) were classified as potent in the mouse MI model and were shown to be clinically beneficial in DMD patients. Thus, higher levels of β-catenin in these two CAP-1002 lots is consistent with potency classified by the in vivo MI mouse model and with clinical efficacy.

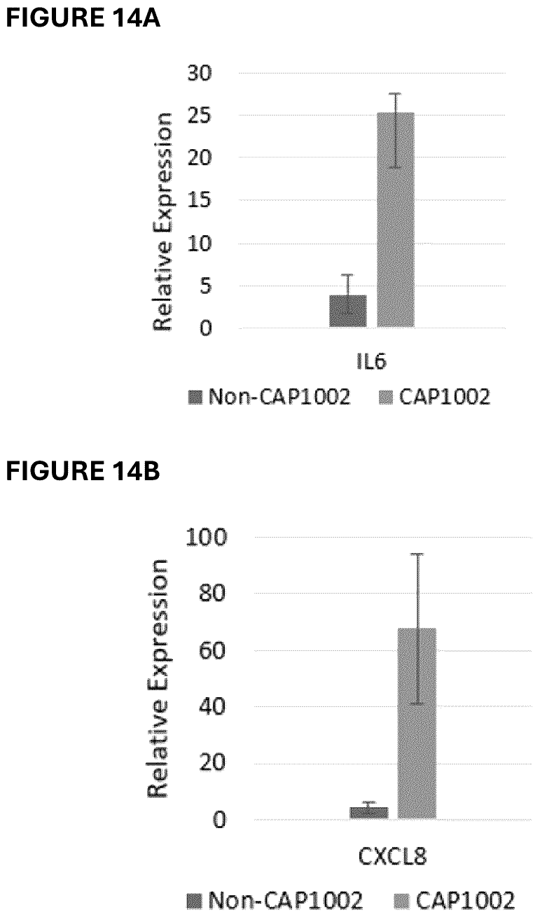

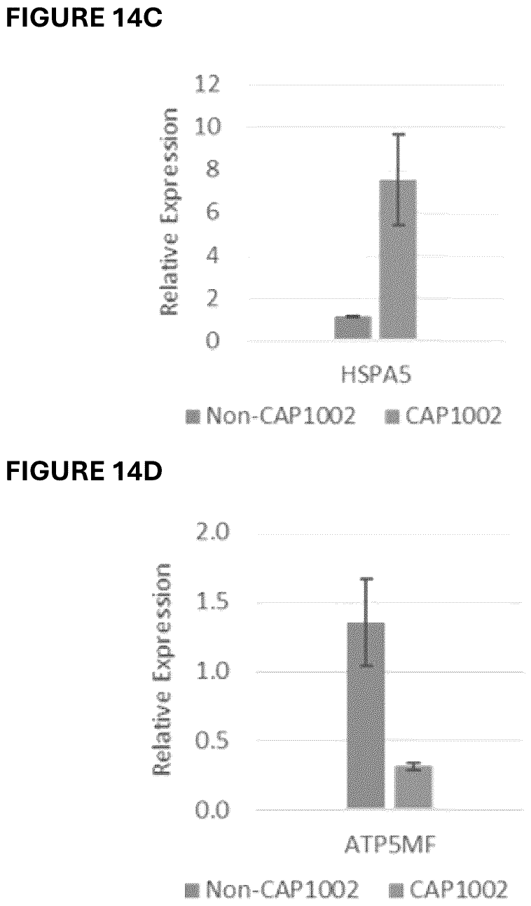

Regarding the identity of the CDC drug product, clinical lots of CAP-1002 were characterized by surface marker expression analyzed by flow cytometry for CD105 (>90%; stem cell marker) and CD45 (<10%; hematopoietic cells) and by cell viability (>70%, reported viable cell number). See FIGS. 14A-14D. In some embodiments, this data, along with HLA typing, endotoxin analysis, FISH, sterility, and Mycoplasma analysis, were/are used for clinical lot release.

In some embodiments, additional assays were performed on clinical lots of CAP-1002 to further characterize these cells including morphological assessment, evaluation of growth characteristics including cell recovery and total population doublings, and analysis of additional surface markers by flow cytometry for CD90, CD140b, CD31 and DDR2.

RNA sequencing is a powerful method to analyze gene expression that can be used to create a cell profile or unique “fingerprint” for varying cell types. The expression profiles of different cells vary. Thus, these expression profiles can be used to differentiate various cell types (i.e., CAP-1002 vs non-CAP-1002 for product identity) and to classify unknown cells (i.e., unknown lots of CAP-1002 vs clinically effective CAP-1002 lots for product potency).

To create a CAP-1002 cell profile, the RNA of 25 CAP-1002 lots were sequenced and used to begin to establish a bioinformatics model to classify the potency of each lot for future product release (Table 4). Of the 25 CAP-1002 lots, LINE B-L1002 is defined as a clinically potent lot since it was used in the HOPE-2 trial where efficacy was demonstrated in DMD patients (McDonald, et. al., 2022). Lots LINE B-L1001, LINE A-L1002, and LINE A-L1004 were “assumed” potent lots since they were produced from the same MCBs as other lots used in the HOPE-2 trial that showed clinical efficacy. Lots from these MCBs were also previously classified as potent using the mouse MI model. In addition, the RNA of 7 non-CAP-1002 cells was sequenced for use as negative (and assumed non-potent) controls in the cell profile model and to determine CAP-1002 identity.

Briefly, cell pellets were prepared for RNA isolation, mRNA library preparation, and next generation sequencing (NGS) to quantify the RNA/transcript expression levels. Bioinformatics was applied to the sequencing data to calculate mRNA abundance and average normalized read counts (NRCs; average normalized value for each gene/transcript). The average NRC for each transcript was calculated for the 4 potent CAP-1002 lots (1 clinically potent lot+3 assumed potent lots) and was then compared to unknown CAP-1002 lots and non-CAP-1002 cells to assess similarity among each sample. A coefficient of variation was calculated for the 4 potent CAP-1002 lots (1 clinically potent lot+3 assumed potent lots) to assess the similarity with a CV of <0.15 included in the analysis, resulting in 207 top transcripts that were classified as similar in the 4 potent CAP-1002 lots by which all other samples were compared.

As shown in Table 4, the 4 potent CAP-1002 lots (1 clinically potent lot+3 assumed potent lots; highlighted green) ranked 1, 2, 5, and 7 with >99% similarity using the top 207 transcripts. The profiles of the other 20 CAP-1002 lots, as analyzed by RNA sequencing, were >95% similar to the 4 potent CAP-1002 lots. One lot of CAP-1002 cells (LINE I-L1004) ranked 28th and was only 87% similar to the 4 potent CAP1002 lots, indicating that this lot may be considerably different than other CAP-1002 lots.

Conversely, the 7 non-CAP-1002 cells used as negative controls (assumed non-potent; highlighted red) ranked 20, 26, 27, 29-32, indicating non-CAP-1002 cells as being substantially different to the 4 potent CAP-1002 lots. The non-CAP1002 cells, including cells of cardiac origin (aortic endothelial cells, aortic muscle cells, cardiac fibroblasts) and non-cardiac origin, ranged from 35-94% similar to the 4 potent CAP-1002 lots. Interestingly mesenchymal stem cells (MSCs) ranked 20th with 96.6% similarity to the 4 potent CAP-1002 lots, and thus, the correlation coefficient of MSCs may be important for setting specifications of CAP-1002 potency (additional MSC sequencing is required to aid in setting specifications).

The gene expression profiles of potent CAP-1002 lots (1 clinically potent lot+3 assumed potent lots; highlighted green) were >99% similar when using the top 207 transcripts for analysis identified by RNA sequencing. The profiles of the other 20 CAP-1002 lots were >95% similar to the 4 potent CAP-1002 lots. Non-CAP-1002 cells (highlighted red) were identified as being substantially different to the 4 potent CAP-1002 lots.

These data demonstrate that the cellular expression profile can be used to differentiate various cell types (i.e., CAP-1002 vs non-CAP-1002 for product identity) and to classify unknown CAP-1002 lots by comparing them to clinically effective, potent CAP-1002 lots for product potency.

TABLE 4

| | | Pearson | |
|---|---|---|---|
| | | Correlation | Similarity |
| Sample Description | Classification | Coefficient | Ranking |
| LINE A, lot 4 | Potent^{a} | 0.9992 | 1 |
| LINE B, lot 1 | Potent^{a} | 0.9978 | 2 |
| LINE D-L1001 | unknown | 0.9969 | 3 |
| LINE E-L1002 | unknown | 0.9963 | 4 |
| LINE B-lot 2 | Potent^{a} | 0.9958 | 5 |
| LINE D-L1002 | unknown | 0.9925 | 6 |
| LINE A, lot 2 | Potent^{a} | 0.9916 | 7 |
| LINE F-L0005 | unknown | 0.9903 | 8 |
| LINE F-L1002 | unknown | 0.988 | 9 |
| LINE G-L1002 | unknown | 0.9847 | 10 |
| LINE H-L1004 | unknown | 0.9816 | 11 |
| LINE I-L1005 | unknown | 0.9806 | 12 |
| LINE F-L2001 | unknown | 0.9796 | 13 |
| LINE J-L1001 | unknown | 0.9794 | 14 |
| LINE F-L2002 | unknown | 0.9792 | 15 |
| LINE K-L1018 | unknown | 0.9783 | 16 |
| LINE L-L1001 | unknown | 0.9734 | 17 |
| LINE G-L1004 | unknown | 0.9691 | 18 |
| LINE L-L1004 | unknown | 0.9666 | 19 |
| Human mesenchymal stem | Non-potent^{a} | 0.9663 | 20 |
| cells (MSC-1) | | | |
| LINE L-L1003 | unknown | 0.9626 | 21 |
| LINE M-L1016 | unknown | 0.9575 | 22 |
| LINE E-L1001 | unknown | 0.9518 | 23 |
| LINE F-L1001 | unknown | 0.9481 | 24 |
| LINE L-L1003 | unknown | 0.9476 | 25 |
| Human aortic endothelial cells | Non-potent^{a} | 0.9393 | 26 |
| (HAEC-1) | | | |
| Human dermal fibroblasts | Non-potent^{a} | 0.8846 | 27 |
| (HDF-1) | | | |
| LINE I-L1004 | Non-potent^{a} | 0.8747 | 28 |
| Human aortic smooth muscle | Non-potent^{a} | 0.834 | 29 |
| cells (HASMC-1) | | | |
| Human atrial cardiac | Non-potent^{a} | 0.6799 | 30 |
| fibroblasts (HACF-1) | | | |
| HEK293 | Non-potent^{a} | 0.6231 | 31 |
| THP-1 | Non-potent^{a} | 0.3519 | 32 |

Transcript data from the initial RNA sequencing study was used to identify a gene signature for CAP-1002 (and non-CAP-1002 cells) as described above. This transcriptome profile was then used to create a heat map of genes that were markedly differentially expressed between CAP-1002 cells and non-CAP-1002 cells (including cells of cardiac origin and non-cardiac origin) making these prime mRNA candidates to differentiate between CAP-1002 cells and other cell types.

Differentially expressed genes were then analyzed by quantitative RT-PCR (qRT-PCR) to establish a quantitative assay to characterize CAP-1002 cells. Briefly, the RNA of 14 CAP-1002 lots (including LINE B-L1002 which is defined as a clinically potent lot since it was used in the HOPE-2 trial where efficacy was demonstrated in DMD patients (McDonald, 2022) and lots LINE B-L1001, LINE A-L1002 which were “assumed” potent lots since they were produced from the same MCBs as other lots used in the HOPE-2 trial that showed clinical efficacy) and the RNA of 7 non-CAP-1002 cells was reverse transcribed into cDNA and quantitative PCR was performed using Taqman probes specific to the differentially expressed gene candidates. Data was analyzed (using delta-delta Ct method) to calculate the relative gene fold expression in all samples. The average CAP-1002 relative gene expression was then normalized to the average relative expression of all non-CAP-1002 cells. As shown in FIG. 4, four genes were significantly different between CAP-1002 cells and non-CAP-1002 cells, including IL6, HSPA6, CXCL8, and ATP5MF. ATP5MF was significantly down-regulated in all CAP-1002 cells by ˜5-fold compared to non-CAP-1002 cells. Conversely, IL6, HSPA5, and CXCL8 were significantly up-regulated in CAP-1002 cells by ˜7-, 8-, and 17-fold, respectively, compared to non-CAP-1002 cells (data averaged for all lots/cells evaluated).

Interestingly, IL6, HSPA5, and CXCL8 (IL8) are known to play a role in anti-inflammation, which is one of the predicted mechanisms of action for CAP-1002.

In one embodiment, the acceptance criterion for therapeutic potency was set at a Pearson Correlation Coefficient of ≥91.0%. Here, it was further shown that 100% of known potent CAP-1002 lots ranked higher (above the 91% Pearson Correlation Coefficient threshold), and 100% of tested non-CAP-1002 cells fell below the threshold using the most stringent and conservative potency model to a significance of p<0.00001. The non-CAP-1002 cells included human cardiac endothelial cells, human dermal fibroblasts, human cardiac fibroblasts, human cardiac smooth muscle, human aortic smooth muscle cells, human atrial cardiac fibroblasts, HEK293 cells, and THP-1 cells (human monocytes).

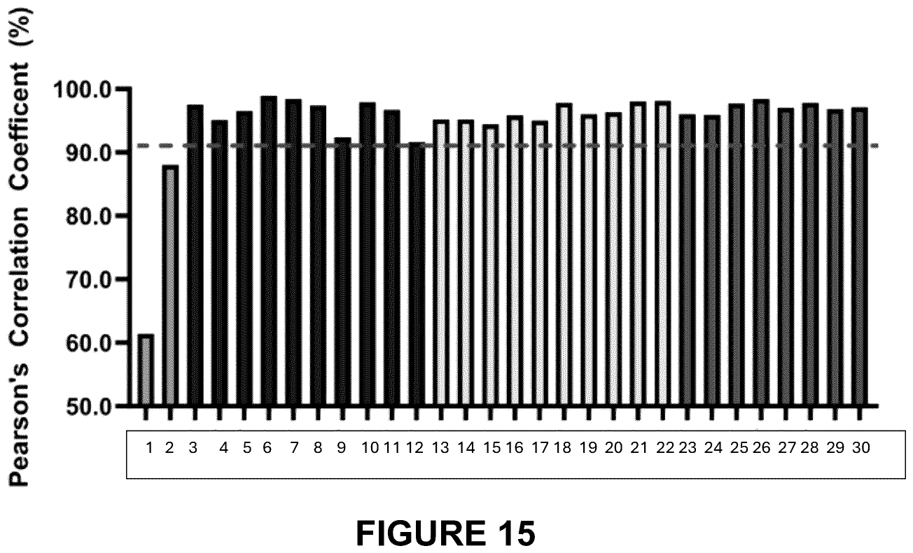

The same 28 lots representing 5 therapeutically potent CAP-1002 lines as demonstrated in the anti-fibrosis collagen assay were assessed according to the RNAseq correlation assay. As shown in FIG. 15, 100% of the CAP-1002 lots exceeded the Pearson Correlation Coefficient acceptance criterion on at least 91%.

In one embodiment, certain CDC culture conditions were shown to produce sub-potent lots. Those conditions include CDC media with ≤5% serum, expanded culture duration, no humidity and normoxia, and no humidity and hypoxia.

In another aspect, the cryopreserved manufactured therapeutically potent CDC doses (a.k.a. CAP-1002 or deramiocel) are shipped to an infusion site (e.g., clinic, hospital, or other approved site where intravenous injections can be performed and which are capable of managing and stored cryopreserved therapeutic cells), thawed, and administered by intravenous infusion to patients with degenerative skeletal or cardia muscle disease, such as, e.g., muscular dystrophy. In one embodiment, a dose is about 150 million CDCs. In one embodiment, a patient in need is administered one dose about every 3 months.

### EXAMPLE

In a specific embodiment, therapeutically potent CDCs are manufactured from human heart tissue. For example, in several embodiments, after receiving donor cardiac tissue and making a gross dissection to produce manageable tissue fragments, an automated dermatome is used in some embodiments, to make an initial cut, for example in the z-axis. In several embodiments, this initial incision is used to cut fragments of cardiac tissue ranging from about 0.2 to about 1.5 g (to be used for a single culture dish). In some embodiments, the initial fragment ranges from about 0.2 to about 0.3 g, about 0.3 to about 0.4 g, about 0.4 to about 0.5, about 0.5 to about 0.6 g, about 0.6 to about 0.8 g, about 0.8 to about 1.0 g, about 1.0 to about 1.2 g, about 1.2 to about 1.5 g, and overlapping ranges thereof. After the initial fragment is generated, cuts in the x-axis and the y-axis are made to generate a cube of tissue (e.g., an explant). In several embodiments, the explant size varies depending on the subtype of tissue, while in other embodiments, a constant explant size is used for the entire donor tissue, regardless of tissue subtype. In some embodiments, the explant ranges from about 100 to about 200 μm3, about 200 to about 300 μm3, about 300 to about 400 μm3, about 400 to about 500 μm3, about 500 to about 600 μm3, about 600 to about 700 μm3, about 700 to about 800 μm3, about 800 to about 900 μm3, about 900 to about 1000 μm3, and overlapping ranges thereof. In some embodiments, the explant size is determined based on the quality of the tissue (e.g., fresh donor tissue versus aged donor tissue). In some embodiments, the explant size is selected to improve the efficiency of downstream steps, including but not limited to enzymatic treatment of the explant (e.g., due to more even penetration of the enzyme into the central portion of the explant).

In several embodiments, the resultant explant is moved from the dissection dish to a culture dish by flooding the explant with culture media and allowing it to come to rest in the culture vessel (as opposed to hand-placement of the explant). Thereafter the explant can be cultured undisturbed for a period of days, and in the interim, additional donor cardiac can be processed in a timely fashion. In some embodiments, the methods disclosed herein enable the use of one or more robotic systems in one or more steps of the process, including, but not limited to, automated flask processing stations. Thus, in some embodiments, human error is reduced, as is, in some embodiments risk of contamination of the culture. The overall scheme for processing and generation of a master cell bank is shown generally in FIG. 1.

Subsequent to the processing of the donor tissue discussed above, a cardiac tissue explant is cultured undisturbed for a period of days in suitable culture media. In several embodiments, the lack of perturbation allows the explant to adhere to the surface of the culture dish (which in several embodiments is coated with a basement membrane-like material such as, for example, laminin, fibronectin, poly-L-orinthine, or combinations thereof) more effectively, which in turn allows the more rapid and robust generation of cells for harvesting. In several embodiments, the culture flasks are treated such that coatings are not necessary (e.g., the explants can, in some embodiments, be cultured in the absence of fibronectin, etc.).

The tissue explants are cultured until a layer of stromal-like cells arise from the adherent explants. This phase of culturing is further identifiable by small, round, phase-bright cells that migrate over the stromal-cells. In certain embodiments, the explants are cultured until the stromal-like cells grow to confluence. At or before that stage, the phase-bright cells are harvested. In certain embodiments, phase-bright cells are harvested by manual methods, while in others, enzymatic digestion, for example trypsin (or a non-animal derived equivalent enzyme), is used. These harvested cells (which are termed Explant-Derived Cells, or EDCs) can then be used to generate cardiospheres, CDCs, frozen for later generation of cardiospheres or CDCs, or subjected to various quality control analyses. Additional information regarding generation of cardiospheres and CDCs, may be found, for example in U.S. patent application Ser. No. 10/567,008, filed Jul. 13, 2006; Ser. No. 11/666,685, filed Apr. 21, 2007; and Ser. No. 13/412,051, filed Mar. 5, 2012, the entireties of each of which are incorporated by reference herein.

In several embodiments, the size of the culture vessels selected to receive the sized explant is varied. In some embodiments, the surface area of the culture vessel is selected to allow one or more explants to be adhered within a single vessel. In some embodiments, the surface area allotted to an explant ranges from about 200 to about 300 cm2, about 300 to about 400 cm2, about 400 to about 500 cm2, about 500 to about 600 cm2, about 600 to about 700 cm2, about 700 to about 800 cm2, about 800 to about 900 cm2, about 900 to about 1000 cm2, about 1000 to about 1100 cm2, about 1100 to about 1200 cm2, and overlapping ranges thereof. In several embodiments, a range between about 400 cm2 and 450 cm2, about 450 cm2 and 500 cm2, about 500 cm2 and 550 cm2, about 550 cm2 and 600 cm2, about 600 cm2 and 550 cm2, about 650 cm2 and 700 cm2, and overlapping ranges thereof (per explant) is used. In some embodiments, commercial culture vessels are used, while in other embodiments, custom vessels are generated.

In several embodiments, particular surface area dedicated to a single explant allows improved growth of cells from the implant. In some embodiments, this is due to reduced contact inhibition or other type of growth inhibition from cells arising from other explants. In some embodiments, the density of resultant cells enables sufficient cell-cell interaction (contact, paracrine, or otherwise) without overgrowth of the cells. In addition to overall improved yield, this also improves the predictability of cell growth such that cell harvesting can be optimized (e.g., avoiding undergrowth or overgrowth). Utilizing the methods disclosed herein, a large number of cells can be generated from the donor tissue. In several embodiments (as compared to the per gram amount of starting tissue) the number of cells generated as a result of the methods disclosed herein is about 1×10\hat{ }5, about 2×10\hat{ }5, about 4×10\hat{ }5, about 6×10\hat{ }5, about 8×10\hat{ }5, about 1×10\hat{ }6, about 2×10\hat{ }6, about 4×10\hat{ }6, about 10×10\hat{ }6, about 20×10\hat{ }6, about 30×10\hat{ }6, about 35×10\hat{ }6, about 40×10\hat{ }6, about 1×10\hat{ }7, about 1×10\hat{ }8, or greater, depending on the embodiment. Thus the ratio of starting tissue mass, based on the unexpectedly advantageous expansion of clinical quality cells based on the methods herein, to clinical doses is about 1:4, about 1:5, about 1:6 about 1:7, about 1:8, about 1:9, about 1:10, and in some cases about 1:20, or greater. Thus, a starting material mass of about 24 grams of cardiac donor tissue will yield about 30 cryovials of explant derived cells, which is suitable, depending on the dose, for approximately 150 patient therapeutic doses.

In addition to the dedicated surface area allotted to an explant, in several embodiments, the subtypes of cardiac tissue derived from the donor sample are optimized. Certain regions of cardiac tissue exhibit distinct characteristics of growth when subjected to the processing described herein. For example, in several embodiments, atrial explants exhibit a rapid cell growth, such that a culture vessel becomes confluent (ready for harvest) prior to explants from other regions. Interestingly, other regions exhibit different growth patterns. See for example, FIGS. 2A-2B. In some embodiments, the various characteristics of the different regions can be exploited in a single culture format, e.g., the explants from multiple regions can be combined (e.g., cultured together) to allow the synergistic interplay between the explants and cells, thereby resulting, in several embodiments, unexpectedly further enhanced growth (as compared to growth of cells from any region alone). For example, in several embodiments, atrial explants are combined with one or more explants from other cardiac regions, for example the right ventricle, septum, left ventricle, or apex and cultured together. In some embodiments, the ratio of mass of the first region is tailored with respect to the mass of the second region. For example, in some embodiments, the ratio is about 1:1, about 1:2, about 1:3, about 1:4, about 1:5, about 1:6, about 1:10, about 1:20 about 20:1, about 10:1, about 6:1, about 5:1, about 4:1, about 3:1, about 2:1, and other ratios within those listed above. In some embodiments, the amount of a first tissue (e.g., atrial to septal, atrial to ventricular, ventricular to septal, atrial to apex, apex to septal, etc.) is about 5% of the amount of the second subtypes, about 10% of the amount of the second subtype, about 15% of the amount of the second subtype, about 20% of the amount of the second subtype, about 25% of the amount of the second subtype, about 30% of the amount of the second subtype, about 35% of the amount of the second subtype, about 40% of the amount of the second subtype, about 45% of the amount of the second subtype, about 50% of the amount of the second subtype, or overlapping ranges thereof. In some embodiments, other ratios or combinations are used. Selection of the ratio is based, in some embodiments, on the status (e.g., quality and/or amount) of the donor tissue. In several embodiments, there is a synergistic communication between the various cells growing (e.g., contact or paracrine) allowing the unexpected increased overall yield and/or rate of cell generation. In several embodiments, various extracellular matrix proteins deposited by the different cells promote the growth and/or viability of cells from a different tissue subtype. In still additional embodiments, other protein-protein interactions occur between the cell types to benefit the growth of the combination of cells. Moreover, as the master cell bank is, in several embodiments, a combination of cells harvested off of each of a plurality of implants, the combination of two (or more) cardiac tissue subtypes yields a more consistent end stem cell batch.

What is claimed is:

1. A method of producing therapeutic cells comprising:
a. selecting a human heart;
b. culturing explant tissue from the selected human heart;
c. outgrowing explant-derived cells (EDCs) from the explanted tissue;
d. harvesting the EDCs;
e. producing cardiospheres by culturing the harvested EDCs under low attachment conditions;
f. collecting the cardiospheres from the low attachment culture;
g. producing cardiosphere-derived cells (CDCs) by seeding and culturing the cardiospheres under high attachment conditions;
h. harvesting the CDCs;
i. cryopreserving the CDCs; and
j. testing a quality control aliquot of the cryopreserved CDCs for identity, purity, potency, post-thaw viability, and cell number; and
k. releasing a therapeutic aliquot for therapeutic use.

2. The method of claim 1, wherein selecting a human heart comprises confirming the eligibility of a donor of the human heart.

3. The method of claim 2, wherein an eligible donor is no more than 60 years old and has an HLA serotype selected from the group consisting of A, B, BW4, C, DR, DR51, DR53, DQB1, and DQA1.

4. The method of claim 2, wherein an eligible donor is free of detectable HIV, HCV, HBV, and syphilis and does not have a medical history of any one or more of transmissible spongiform encephalopathy, zika virus, vaccinia, chagas disease, and sepsis.

5. The method of claim 1, wherein the explant tissue is prepared by a method comprising dissecting the heart; creating approximate 500-micron cubes; and seeding about 500 mg of tissue per 500-2,000 square cm of tissue culture surface.

6. The method of claim 1, wherein the EDCs are cultured by a method comprising incubating the explants for several days; then adding media after several days; exchanging media every several days; and harvesting the EDCs at about 80% cell confluency.

7. The method of claim 6 further comprising pooling the harvested EDCs obtained from a single donor heart; centrifuging the pooled EDCs into a pellet; formulating the EDCs with a cryopreservative; aliquoting the formulated EDCs into master cell bank (MCB) and quality control (QC) vials; and freezing the MCB and QC aliquots.

8. The method of claim 7, wherein (i) the pooled EDCs are tested for viability, Mycoplasma, and HLA and (ii) the MCB or QC aliquots are subjected to flow cytometry and assessed for sterility and the presence of viral agents.

9. The method of claim 6 further comprising thawing an MCB cell aliquot; washing the MCB cells to remove the cryopreservative; seeding and the washed MCB cells in media onto a low-attachment substrate; and incubating the seeded MCB cells for several days, wherein cardiospheres are produced.

10. The method of claim 1, wherein the high attachment conditions comprise culturing the cardiospheres or CDCs on a fibronectin-coated tissue culture surface.

11. The method of claim 1 comprising expanding the CDCs by passaging the CDCs every 2-7 days or at 70%-100% confluency, wherein the CDCs are seeded at about 5,000-10,000 cells per square centimeter of cell culture surface.

12. The method of claim 10 further comprising harvesting the expanded CDCs at about 80%-90% confluency, wherein the harvesting comprises (i) detaching the CDCs from the fibronectin-coated tissue culture surface; (ii) resuspending the detached CDCs in media containing 25% human albumin; (iii) filtering the resuspended CDCs; and (iv) washing the filtered CDCs with phosphate buffered saline (PBS).

13. The method of claim 1, wherein the cryopreserving of the selected CDCs comprises (i) formulating washed CDCs with a hypothermic solution comprising one or more of a buffer, a sugar, a sugar alcohol, glutothione, one or more free-radical scavengers, and human albumin at a concentration of about 18 million cells per milliliter; (ii) assessing cell viability; (iii) adding a serum-free protein-free solution containing 10% DMSO to a cell suspension concentration of about 9 million cells per milliliter; (iv) filling the cell suspension into vials, labeling, and stoppering; and (v) freezing the filled vials by controlled rate freezing.

14. The method of claim 1, wherein the identity of the therapeutic is determined by measuring the expression of IL6, HSPA5, CXCL8, and CD105 in a quality control aliquot of the CDCs.

15. The method of claim 14, wherein the identity or purity of the CDCs is confirmed when (i) IL6 mRNA is expressed at least 140-fold compared to human dermal fibroblasts as determined by qRT-PCR, (ii) HSPA5 mRNA is expressed at least 5-fold compared to human dermal fibroblasts as determined by qRT-PCR, (iii) CXCL8 mRNA is expressed at least 20-fold compared to human dermal fibroblasts as determined by qRT-PCR, (iv) greater than 90% of the CDCs express CD105 as determined by flow cytometry, or (v) less than 10% of the CDCs express CD45 as determined by flow cytometry.

16. The method of claim 1, wherein post thaw viability of the CDCs is at least 70%

17. The method of claim 1, wherein the potency of the CDCs is confirmed by (i) RNAseq transcriptome fingerprint correlation to a standard therapeutic transcriptome fingerprint, and (ii) an anti-fibrosis assay.

18. The method of claim 17, wherein the CDCs are considered potent and are selected for therapeutic use when the RNAseq transcriptome fingerprint is at least 91% correlated to the standard therapeutic transcriptome fingerprint as determined by Pearson's correlation coefficient.

19. The method of claim 17, wherein the anti-fibrosis assay comprises contacting fibroblasts with CDC conditioned media; extracting RNA from the contacted fibroblasts and performing quantitative reverse transcription polymerase chain reaction (qRT-PCR) specific to type I collagen (COL1A) and specific to type III collagen (COL3A); comparing COL1A expression and COL3A expression from the fibroblasts contacted with the CDC conditioned media to COL1A and COL3A expression from control fibroblasts contacted with non-conditioned media; and selecting for therapeutic use those CDCs that provided conditioned media that inhibited COL1A expression by at least 35% relative to control and that inhibited COL3A expression by at least 45% relative to control.

20. A cell therapy composition comprising therapeutic cells produced according to claim 1.

21. A method of treating dystrophic muscle in a subject in need thereof comprising
a. selecting a human heart;
b. culturing explant tissue from the selected human heart;
c. outgrowing explant-derived cells (EDCs) from the explanted tissue;
d. harvesting the EDCs;
e. producing cardiospheres by culturing the harvested EDCs under low attachment conditions;
f. collecting the cardiospheres from the low attachment culture;
g. producing cardiosphere-derived cells (CDCs) by seeding and culturing the cardiospheres under high attachment conditions;
h. harvesting the CDCs;
i. cryopreserving the CDCs; and
j. testing a quality control aliquot of the cryopreserved CDCs for identity, purity, potency, post-thaw viability, and cell number;
k. releasing a therapeutic aliquot of the cryopreserved CDCs for therapeutic use.
l. shipping the cryopreserved CDCs to an infusion site facility;
m. thawing the cryopreserved CDCs; and
n. administering a therapeutic dose of thawed CDCs to the subject.

22. The method of claim 21, wherein selecting a human heart comprises confirming the eligibility of a donor of the human heart, wherein an eligible donor (i) is no more than 60 years old, (ii) has an HLA serotype selected from the group consisting of A, B, BW4, C, DR, DR51, DR53, DQB1, and DQA1, (iii) is free of detectable HIV, HCV, HBV, and syphilis, and (iv) does not have a medical history of any one or more of transmissible spongiform encephalopathy, zika virus, vaccinia, chagas disease, and sepsis.

23. The method of claim 21, wherein the explant tissue is prepared by a method comprising (a) dissecting the heart; creating approximate 500-micron cubes; (b) seeding about 500 mg of tissue per 500-2,000 square cm of a tissue culture surface; (c) incubating the explants for several days in media; (d) exchanging the media every several days; (e) harvesting the EDCs at about 80% cell confluency; and pooling the harvested EDCs obtained from a single donor heart.

24. The method of claim 23 comprising seeding the pooled EDCs onto a low-attachment substrate in media; incubating the seeded EDCs cells for several days, wherein cardiospheres are produced; culturing the cardiospheres on a high attachment substrate, wherein the high attachment substrate comprises a fibronectin-coated tissue culture surface and wherein CDCs are produced.

25. The method of claim 21 comprising (a) expanding the CDCs by passaging the CDCs every 2-7 days or at 70%-100% confluency, wherein the CDCs are seeded at about 5,000-10,000 cells per square centimeter of cell culture surface; and (b) harvesting the expanded CDCs at about 80%-90% confluency, wherein the harvesting comprises (i) detaching the CDCs from the fibronectin-coated tissue culture surface; (ii) resuspending the detached CDCs in media containing 25% human albumin; (iii) filtering the resuspended CDCs; and (iv) washing the filtered CDCs with phosphate buffered saline (PBS).

26. The method of claim 21, wherein the cryopreserving of the selected CDCs comprises (i) formulating washed CDCs with a hypothermic solution comprising one or more of a buffer, a sugar, a sugar alcohol, glutothione, one or more free-radical scavengers, and human albumin at a concentration of about 18 million cells per milliliter; (ii) assessing cell viability; (iii) adding a serum-free protein-free solution containing 10% DMSO to a cell suspension concentration of about 9 million cells per milliliter; (iv) filling the cell suspension into vials, labeling, and stoppering; and (v) freezing the filled vials by controlled rate freezing.

27. The method of claim 21, wherein the identity of the therapeutic is determined by measuring the expression of IL6, HSPA5, CXCL8, and CD105 in a quality control aliquot of the CDCs.

28. The method of claim 27, wherein the identity or purity of the CDCs is confirmed when (i) IL6 mRNA is expressed at least 140-fold compared to human dermal fibroblasts as determined by qRT-PCR, (ii) HSPA5 mRNA is expressed at least 5-fold compared to human dermal fibroblasts as determined by qRT-PCR, (iii) CXCL8 mRNA is expressed at least 20-fold compared to human dermal fibroblasts as determined by qRT-PCR, (iv) greater than 90% of the CDCs express CD105 as determined by flow cytometry, or (v) less than 10% of the CDCs express CD45 as determined by flow cytometry.

29. The method of claim 21, wherein post thaw viability of the CDCs is at least 70%

30. The method of claim 21, wherein the potency of the CDCs is confirmed by (i) RNAseq transcriptome fingerprint correlation to a standard therapeutic transcriptome fingerprint, and (ii) an anti-fibrosis assay.

31. The method of claim 30, wherein the CDCs are considered potent and are selected for therapeutic use when the RNAseq transcriptome fingerprint is at least 91% correlated to the standard therapeutic transcriptome fingerprint as determined by Pearson's correlation coefficient.

32. The method of claim 30, wherein the anti-fibrosis assay comprises contacting fibroblasts with CDC conditioned media; extracting RNA from the contacted fibroblasts and performing quantitative reverse transcription polymerase chain reaction (qRT-PCR) specific to type I collagen (COL1A) and specific to type III collagen (COL3A); comparing COL1A expression and COL3A expression from the fibroblasts contacted with the CDC conditioned media to COL1A and COL3A expression from control fibroblasts contacted with non-conditioned media; and selecting for therapeutic use those CDCs that provided conditioned media that inhibited COL1A expression by at least 35% relative to control and that inhibited COL3A expression by at least 45% relative to control.

33. The method of claim 21, wherein a therapeutic dose comprises 150 million CDCs.

34. The method of claim 33 comprising administering the therapeutic dose to the subject by intravenous infusion about every 3 months.

35. The method of claim 21, wherein the subject in need suffers from a degenerative muscle disease.

36. The method of claim 35, wherein the subject in need has muscular dystrophy.

37. The method of claim 36, wherein the subject in need has Duchenne muscular dystrophy.
